(19) United States
(12) Patent Application Publication
(10) Pub. No.: US 2017/0014297 A1
(43) Pub. Date: Jan. 19, 2017

(54) EXOSKELETON SLIPPER

(71) Applicant: ROBOTIQUES 3 DIMENSIONS, Auxerre (FR)

(72) Inventors: Serge Grygorowicz, Gy l'Eveque (FR); Aurélie Riglet, Dammarie sur Loing (FR); Antoine Perrin, Saulxures sur Moselotte (FR)

(73) Assignee: ROBOTIQUES 3 DIMENSIONS, Auxerre (FR)

(21) Appl. No.: 15/124,234

(22) PCT Filed: Mar. 10, 2015
(86) PCT No.: PCT/FR2015/050593

§ 371 Date: Sep. 7, 2016

(30) Foreign Application Priority Data
Mar. 11, 2014 (FR) 1451981

(51) Int. Cl. A61H 3/00; A61B 5/103; A43B 3/00; A43B 7/32; A43C 19/00; B25J 9/00; A61B 5/11

(52) U.S. Cl. CPC A61H 3/00; B25J 9/0006; A61B 5/1036; A61B 5/112; A61B 5/1123; A43B 7/32; A43C 19/00; A43B 3/0005; A61H 2205/12; A61H 2003/001

(57) ABSTRACT

A slipper, to an exoskeleton, and to a method for using such a slipper is disclosed. The slipper includes means for attaching said slipper to a shoe or foot of a user, and a rigid sole for supporting the load to the ground that is hingeably connectable to the end of a leg of the exoskeleton. The attachment means are attached to and hinged about a pivoting axis secured to the rigid sole on the side of the distal end thereof, such as to enable the heel of the foot or the shoe of the user to rise from the rigid sole when walking.

The present invention relates to an exoskeleton slipper comprising means for fixing to a shoe or a foot of a user, and a rigid sole which supports the load to the ground and is able to be connected in an articulated manner to the end of a leg of the exoskeleton.

It also relates to an exoskeleton provided with such slippers and to a corresponding method of use.

It is used particularly advantageously, but not exclusively, in the field of walking exoskeletons, that is to say exoskeletons used for carrying loads or in the field of assisting a maneuver.

However, it can also be used in training or in rehabilitation with the aim of recovering a natural gait or of achieving a faster walking speed.

At present, walking with an exoskeleton requires the feet to he kept permanently flat in an almost automatic manner, that is to say it is not possible to lift the heel of the operator while the foot in the aerial phase is not totally supported unless fighting against the exoskeleton. In this case, it is the ankle of the operator that has to provide the flexion/extension ankle moment necessary for taking up the stress of the exoskeleton, which causes discomfort and excessive expenditure of energy for the operator.

Exoskeletons are thus known (EP 2 296 602) in which the sole of the exoskeleton is coupled to the shoe or the foot of the operator. This sole, joined to the leg of the exoskeleton bearing on the ground, does not permit any degree of freedom between the sole of the exoskeleton and the shoe/foot of the operator.

At the very most when it is flexible, it allows a movement about the metatarso-phalangeal joint of the foot, on condition of fighting against the exoskeleton bearing on the ground.

Devices are also known (WO 2013188868) in which the weight is not transferred directly to the ground but instead through the structure of the human body, for example in the area of the tibia.

Such systems still have disadvantages here, especially associated with the considerable effort and the high oxygen consumption generated by the user in order to operate these systems.

The present invention aims to make available an exoskeleton slipper, an exoskeleton, and a method for use of such an exoskeleton with slippers, which respond better than those previously known to the practical requirements, especially in the sense that the invention allows more natural walking with an exoskeleton, and it does so with less energy being consumed by the user than is the case with the known exoskeletons.

To do this the invention proceeds from a principle which is entirely different than the one known, namely allowing the foot of the operator to lift away from the sole of the exoskeleton. The exoskeleton is then able to transfer weight to the ground as the human lifts the heel.

It is true that, in doing this, the exoskeleton is not anthropomorphic, that is to say the flexion and extension of the user do not coincide with those of the exoskeleton.

Similarly, the setting in motion of the leg will be performed at the front of the foot, which may appear problematic.

Indeed, with the invention, walking will be more natural. The lifting of the heel in fact facilitates the longest possible strides and therefore allows walking at a more sustained rhythm. It also makes it easier to climb stairs or to adopt a crouched position.

The lifting of the heel also allows the exoskeleton to bear on its leg for as long as possible, which permits better assistance (reduced oxygen consumption).

Moreover, in one embodiment of the invention, and by equipping it for example with a linear potentiometer that detects the lifting of the human heel with respect to the exoskeleton, it will be possible to anticipate the wish of the operator to lift the foot.

A measurement of the lifting of the heel in fact makes it possible to anticipate the aerial phase of the exoskeleton or the contact phase, since the sole of the exoskeleton comes into contact, or not, with the ground before the mobile sole. This permits better acceptance of the slight offset between the exoskeleton and the human, on account of the considerable reactivity that is then possible by virtue of the anticipated command of the function of the exoskeleton.

Finally, with the invention, in the aerial phase, the flexion/extension couple of hip and knee can be proportional to the lift sensor (followed in position around the lifting of the heel), which will make it possible to suppress all the efforts for moving the leg, by virtue of the observation of the intention of the user, for example in two directions.

To this end, the present invention principally proposes an exoskeleton slipper comprising means for fixing to a shoe or a foot of a user, and a rigid sole which supports the load to the ground and is able to be connected in an articulated manner to the end of a leg of the exoskeleton, characterized in that the fixing means are fixed and articulated pivotably about a pivot axis or a pivot point integrally connected to the sole in the area of the distal end thereof, no as to allow the heel of the foot or the shoe of the user to lift from the rigid sole when walking.

Articulated pivotably signifies a rotational connection in particular. However, the latter can also be more generally what is called a universal connection, which will permit pivoting in the transverse or sagittal axis but also a compliance in the longitudinal or frontal axis with respect to the rigid sole, for example by way of a ball joint.

In advantageous embodiments, recourse is also and/or moreover had to one or more of the following arrangements:
the pivot axis is an axis of rotation perpendicular to the longitudinal direction of the sole and situated at one third or substantially at one third of its length from the distal end thereof, so as to coincide or substantially coincide with the axis of the metatarso-phalangeal joint of the foot of the user using the slipper;
the fixing means comprise a second sole fixed at one end to the distal end of the rigid sole and free with respect to said rigid sole at the other end in order to permit the pivoting about the pivot axis, and retention means for holding the foot or the shoe on said second sole;
the fixing means comprise, in the distal part of the rigid sole, means for limiting the angle of pivoting of the second sole to below a defined angle value and/or stiffening means designed to resist the pressure exerted by the heel of the user during functioning. The stiffening means are, for example, a system with a spring of adjustable stiffness, as is known per se;
the fixing means comprise a flexible blade which supports the foot or the shoe of the user and which is fixed to the distal part of the rigid sole at one end and free at the other end, and means for connecting the foot or the shoe of the user to said blade. The flexible blade naturally assumes an angle of several degrees, for example 30°, in the unstressed position and/or comprises means for angle limitation;
the flexible blade has a flexural stiffness of between 1 Nm/rad and 45 Nm/rad;
the shoe of the user being equipped with a male or female clip-on end, the fixing means comprise a female or male clip-on member of complementary shape (designed to cooperate with the shoe end), integrally connected to the distal end part of the rigid sole and movable in rotation about said pivot axis;
the slipper has abutment means designed to limit the rotation about the pivot axis to an adjustable value of less than 30° with respect to the rigid sole;
the slipper comprises a first pressure sensor fixed at the distal part of the rigid sole, and a second pressure sensor situated at the proximal part of said sole, and measuring and calculating means designed to detect the lifting of the foot or of the shoe of the user from said rigid sole on the basis of the measurements carried out on said sensors.

Thus, in the aerial phase, it will be possible to effect position monitoring around the lifting of the heel in order to eliminate or considerably limit the forces that have to be supplied by the operator in order to set the leg in motion.

This will permit what is called a contactless command for setting the exoskeleton in motion, which is comparable to a command effected with an infinitely flexible force sensor.

Indeed, using the sensors of the position of lifting of the heel set in parallel with the spring, the force F=k×Δl is deduced where k is the stiffness of the spring and Δl is the lifting of the heel. The stiffness of the spring (flexible blade) being very low in respect of the forces to be supplied to set the leg in motion, this permits very good observation of the intention. The command is thus made with, as setpoint value, a zero force at the area of the heel.

Another method of anticipating the movement of the exoskeleton can moreover advantageously be used.

Here, the reactivity of the exoskeleton through the “slope” parameter corresponding to the maximum admissible derivative for the observation, the variation in weight from one foot to the other, is attenuated or increased as a function of the lifting of the heels.

In fact, when the two heels are in a bearing position, it is considered highly probable that the operator is stationary. He therefore does not require much reactivity. The slope is therefore parameterized to be low.

When one of the heels is lifted, it is likely that the operator wishes to take a physical step. Here, therefore, the exoskeleton must, on the contrary, be reactive. The slope is then increased as a function of the measurement of lift of the heels.

By contrast, when the two heels are lifted, it is then possible that the operator is in the process of crouching down. This is why it is the absolute value of the difference of the two lifts that is used to vary the slope.

For example, if Δlg and Δld are the values of lift of the left heel and of the right heel, respectively, and p is the slope parameter, this will give:

p=a×|Δlg−Δld|+b

with a: slope variation gain and b: slope value (termed weak).

Finally, it will be noted that the lifting of the heel brings about a supplementary component associated with the pressure exerted on the foot. Indeed, the stiffness of the spring and/or the reaching of the abutment of the shoe applies a pressure force to the ball of the foot (result of the stresses in the connections of the foot: internal coupling).

The lifting then brings about an increase in the pressure observed, whereas this pressure ought to decrease with a view to setting the leg in motion. An identification of these stresses as a function of the lift (linear equation) allows this defect to be eliminated. This defect is not inconsiderable, since it can represent up to 20% of the pressure exerted by the weight of the operator.

Advantageously, the slipper moreover comprises shock-absorbing means (unidirectional spring, for example) designed to absorb the heel strike.

Such a shock absorber makes it possible to eliminate the annoying flip-flop effect at the moment of lifting of the foot (jolting in the shank) due to the mass and inertia of the rigid sole and to the return to position of the spring. The shock absorber will therefore act only in the direction of the heel strike and not during lift.

The invention also relates to an exoskeleton for the lower limbs, comprising two articulated legs, each having a thigh, a shank and means for connecting the lower end of the shank to a slipper as described above, characterized in that the means of connection to the slipper comprise an elongate component of a defined length, articulated in rotation at one end with said lower end of the shank and rigidly fixed to form an angle at the other end with the rigid sole.

This angle is, for example, between 90° and 50°, for example, 80°.

For structural reasons, such an arrangement will bring about articulated legs that are necessarily longer than the sum total of the lengths, placed end to end, and in the continuation of each other, of the thigh and of the shank.

Although a priori contrary to the anthropomorphic arrangements that are intuitively sensed as necessary for correct functioning of an exoskeleton of the lower limbs, such an arrangement in fact does not impede the operator during the walking cycle, the legs of the exoskeleton (thigh+shank) then being arranged to be longer than those of the operator. In practice, the connections on the thighs are thus omitted, in contrast to the exoskeletons of the prior art.

Advantageously, the exoskeleton comprises a connecting member between the upper ends of the legs on which it is articulated, said connecting member being able to be positioned at the level of the pelvis of the user, and means for commanding the actuation of the articulated legs as a function of the measuring and calculating means which are designed to detect the lifting of the foot or of the shoe of the user from said rigid sole on the basis of the measurements carried out on said sensors, such that the lifting of the foot of the user is anticipated.

Advantageously, moreover, it comprises calculating means for limiting the forces of the user on the exoskeleton in the position of lifting of the foot of the user, during movement of the leg.

The invention also relates to a method for using an exoskeleton comprising slippers, as described above.

The invention also relates to a method for using an exoskeleton comprising slippers provided with a rigid sole and means for fixing the feet of a user on said sole, characterized in that the fixing means are movable in rotation about a pivot axis integrally connected to the sole in the area of the distal end thereof, so as to allow the heel of the shoe of the user to lift from the rigid sole when walking.

the user being in the process of walking,

the pressure values and/or the angle values of the heels of the user with respect to the ground are acquired continuously and/or at regular time intervals, and

when the pressure and/or the angle detected drop in value below reference values that are introduced beforehand into the calculating means and correspond to a foot bearing on the ground, a command anticipating the movement of the leg is generated.

Advantageously, the detection of the drop in pressure and/or of the angle corresponding to the detection of an intention of movement, an automatic function mode of the legs of the exoskeleton is initiated, so-called robot mode, using a previously recorded gait pattern.

The recording takes place at the start of use depending on the requirements and in particular concerns the angles, speeds and couples that one wishes to use.

It will thus be possible, in an even more effective manner, to partially eliminate the force necessary for overcoming the inertia of the legs of the exoskeleton, specifically following the angle of the fixing means and/or the pressure differentials with respect to the rigid sole, as a function of the predictive command.

The invention will be better understood on reading the following description of embodiments which are given below as non-limiting examples.

The description makes reference to the drawings which accompany it and in which:

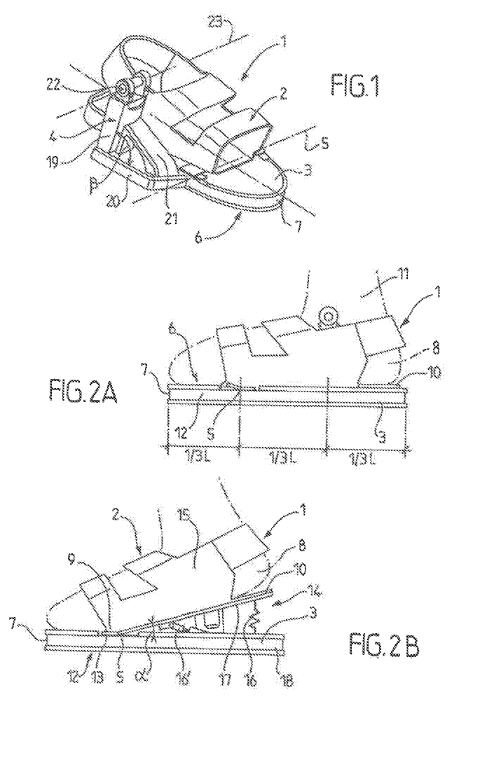

FIG. 1 is a perspective view of a first embodiment of a slipper according to the invention.

FIGS. 2A and 2E are side views of the slipper from FIG. 1, in the bearing position and the lifting position, respectively.

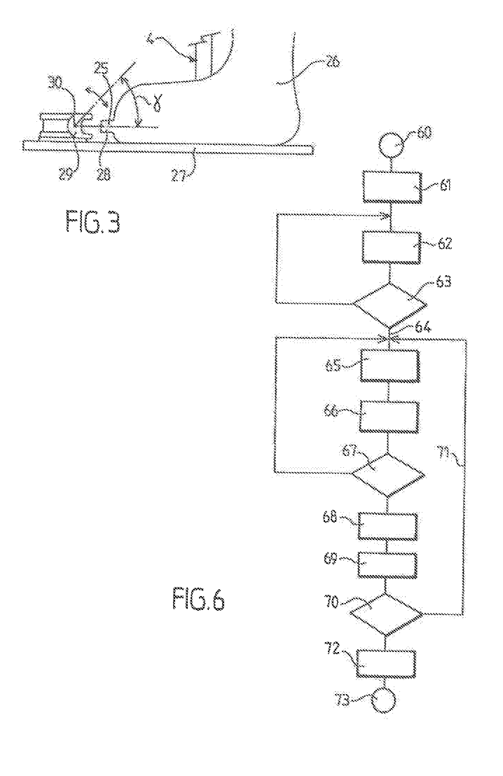

FIG. 3 is a schematic side view showing the principle of another embodiment of the fixing means for articulation about a pivot axis according to the invention.

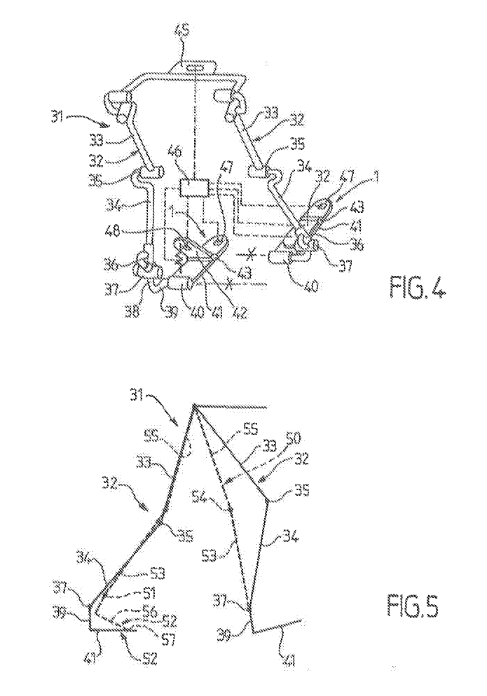

FIG. 4 is a schematic and Perspective rear view of an exoskeleton according to an embodiment of the invention.

FIG. 5 schematically illustrates the relative positions of the exoskeleton with respect to the legs of the user, in the embodiment of the invention more particularly described here.

FIG. 6 shows a flow chart of the main steps of a method for using an exoskeleton, according to an embodiment of the invention.

FIG. 1 shows an exoskeleton slipper comprising means 2 for fixing to a shoe or to a foot of a user (not shown) and a rigid sole 3 able to be connected in an articulated manner by connecting means 4 to the end of a leg of the exoskeleton (not shown).

The rigid sole is, for example, a plate, for example of metal, for example with a thickness of 3 mm and with a shape that is oval in the area of the end of the foot and substantially rectangular in the area of the heel, in a manner known per se in the field of shoes.

The fixing means 2 (cf. also FIGS. 2A and 2B) are fixed and articulated about a pivot axis 5 integral with the sole 3 in the area 6 of the distal end thereof, so as to allow (cf. FIG. 2B) the heel 8 of the shoe and/or of the foot of the user to lift from the rigid sole 3 during walking.

The pivot axis 5 is perpendicular to the longitudinal direction of the sole 3 and is situated substantially at one third of its length L from the distal end 7 thereof, so as to coincide with the metatarso-phalangeal joint 9 of the foot of the user using the slipper 1.

More precisely, the slipper according to the embodiment of the invention described with reference to FIGS. 1, 2A and 2E comprises a flexible blade 10 for supporting the foot 11 of the user, fixed at one end 12, in its distal part 13 situated toward the distal end 7 of the rigid sole 3, and free at the other end 14, in order to permit lifting of the heel.

The fixing means 2 likewise comprise retention means 15 known per se, for example a strap enclosing the rear and top of the foot or of the shoe, with Velcro and/or fastening means known per se.

The flexible blade 10 is, for example, made of spring steel and here has a stiffness of 10 Nm/rad, for example.

Restoring means and/or complementary spring means 16 can also be provided between the heel part 17 of the flexible blade 10 and the proximal end part 18 of the sole 3.

The means 16 are, for example, adjustable in a manner known per se, with limitation of the angle α of lift.

In the embodiment more particularly described here, the connecting means 4 for connection to the end of the shank of the leg of the exoskeleton comprise an elongate component 19, for example of the same height (with respect to the sole) as the rear part of the straps that hold the slipper on the sole.

The component 19 is, for example, formed by a small parallelepipedal tube, integrally connected to the sole 3 in the lower part by way of a rectangular end plate 20. More precisely, it forms part of and comprises reinforcement means 21 giving this tubular element 19 an angle β, for example of 80°, with respect to the sole. The element 19 is terminated in the upper part by an axle 22 for rotational connection to the end of the shank (not shown) in a direction 23 parallel to the pivot axis 5.

FIG. 3 shows schematically another embodiment of a slipper using a connection system of the type used in the field of cross-country skiing and/or of cyclists' shoes, which will permit an articulation in rotation starting from the front end 25 of the shoe 26.

The latter bears on the rigid sole 27, the connection system comprising a male end 28 which attaches to the female member^{.} 29 by way of a ball joint 30 movable in rotation in the sagittal plane between an angle of 0° and a maximum angle Υ of 30°, for example.

The ball joint can also have a degree of freedom in the frontal and/or transverse planes.

Means known per as make it possible to adjust the blocking force and/or frictional force of the ball joint 30.

The rigid sole 27 is moreover connected in a known manner, for example as described with reference to FIG. 1, to means 4 for connection to the end of the shank (not shown).

FIG. 4 shows an exoskeleton 1 comprising slippers 1 according to the embodiment of the invention of the type described with reference to FIG. 1.

The exoskeleton 31 comprises two articulated legs 32, each formed in a manner known per se by two thighs 33 and two shanks 34 which are connected by ball joints 35 actuated in a known manner by a motor. The lower ends 36 of the shanks 34 are connected in rotation at 37 to an end 38 of a rod 39, which is bent here for example, but which can be straight as shown in FIG. 1.

The rod 39 is fixed integrally at its other end. 40 to the proximal end of the rigid sole 41 (the type of sole 3 described with reference to FIG. 1). The part 42 of the second sole of the fixing means is mounted movably in rotation about the axis 43 between a position of contact with the rigid sole and a position of lift.

In the embodiment more particularly described here, the exoskeleton moreover has command means 45 for anticipating the movement actuating the motors (not shown), making it possible to move the thighs and the shanks, the movements for their part taking place in a manner known per se.

These means for commanding the actuation of the articulated legs are connected to the measuring and calculating means 46 formed by a small calculator programmed on the basis of the measurements carried out on the slipper engaged by the foot of the user.

To do this, they comprise a first pressure sensor 47 fixed to the distal part of the rigid sole, and a second sensor 48 fixed either to the proximal part of said sole or to the movable part 42 of the second sole.

These sensors are, for example, weight sensors such as a strain gauge.

The measuring and calculating means 46 for their part are designed to detect the lifting of this movable part, and therefore of the heel of the shoe of the user, on the basis of the difference in the values of measurements carried out on said sensors, and this in a manner known per se.

It will be noted that a linear potentiometer (not shown) can advantageously also be used with or without the sensors, in a manner known per se.

Indeed, a linear potentiometer in particular is on its own able o observe the lifting of the heel by way of an approximation using the rules of trigonometry.

In fact, if α is the angle of the heel to the ground and BC is the distance between the point of articulation and the bearing point of the heel lifted by the height Δ1, this will give Δ1=BC sin(α).

FIG. 5 shows the respective positions of the legs 50 of the user (in broken lines) and of the exoskeleton 31 (in solid lines) during walking, using the same reference numbers to designate the same features as for FIG. 4.

The articulation 37 of the rod 39 is situated substantially at the level of the joint 51 of the foot 52 (when flat) of the user, with his shank 53 itself connected in an articulated manner at 54 to the corresponding thigh 55.

When the sole 41 of the first leg 32 is bearing on the ground, the heel 56 of the user is lifted, the tip 57 of his feet being fixed to the end of the sole 41, which allows him to walk, the second leg 32 of the exoskeleton for its part being in motion and lifted off the ground with the second foot of the user, of which the leg is then in a position held to the front in order to advance during walking (cf. FIG. 5).

There is therefore weight bearing on the right (rear) leg. It will be noted that, so as not to impede the user during his gait cycle, the legs of the exoskeleton (thigh+shank) are longer than those of the user and that the connections between the thighs of the user and the exoskeleton must be omitted.

The steps involved in the use of an exoskeleton. with the slippers according to the invention will now be described with reference to the flow chart in FIG. 6.

This embodiment describes semi-automatic functioning. It goes without saying that it can be made completely automatic.

After the exoskeleton has been initialized and turned on (step 60), the user programs (step 61) the mode of use that he wishes to employ (sustained period of walking, rehabilitation of left leg after accident, etc.).

He then puts on the slippers and adjusts the exoskeleton (step 62). At this stage he carries out certain fine-tuning tests (step 63), which may or may not entail new adjustments.

The exoskeleton is then ready for walking (link 64).

By way of the sensors 47, 48, he then obtains the pressure values of the heels and of the toes of the user with respect to the ground (step 65).

From these values, he then calculates (step 66), by subtraction, if the user has or has not begun to ease the pressure on one foot in order to start transferring said pressure to the other foot, this making it possible to anticipate the intention of the user, and hence the movement of his corresponding leg.

In the case of positive detection (test 57), command of anticipation of the movement is then generated at 68, and the movement of the leg is effected (step 69). If not, the steps 65, 66 are repeated until differential detection.

It should be noted that the command generated at 68 can also call on a specific programmed instruction mode or pattern which generates a physical step adapted to the morphology of the operator and the desired speed (for example gained from experience or the literature in a manner known to a person skilled in the art).

The values of the sensors are also tested (step 70) permanently and/or at repeated defined time intervals (for example every tenth or one hundredth of a second).

When the values obtained make it possible to determine that the following feet are in the initial bearing position, the procedure returns (link 71) to the initial steps 65, 66, etc.

Otherwise, a final step 72 of stabilization is performed before stopping.

As will be appreciated, and as is also apparent from the above, the present invention is not limited to the embodiments more particularly described. Instead, it includes all variants thereof, in particular those in which the retention means on the second sole are different, or those, for persons who are disabled or in rehabilitation, where provision is made to passively activate the lifting movement, for example by way of a preloaded spring as a function of the weight of the user.

In this case, the lifting of the heel would be triggered when the weight of the user is placed on the front of the foot.

1. An exoskeleton slipper comprising means for fixing to a shoe or a foot of a user, and a rigid sole which supports the load to the ground and is able to be connected in an articulated manner to an end of a leg of the exoskeleton, wherein said means for fixing are fixed and articulated pivotably about a pivot axis or a pivot point integrally connected to the rigid sole in an area of the distal end thereof, so as to allow the heel of the foot or the shoe of the user to lift from the rigid sole when walking.

2. The slipper as claimed in claim 1, wherein the pivot axis is an axis of rotation perpendicular to the longitudinal direction of the sole and situated at one third or substantially at one third of its length from the distal end thereof, so as to coincide with the axis of the metatarso-phalangeal joint of the foot of the user using the slipper.

3. The slipper as claimed in claim 2, wherein said means for fixing comprise a flexible blade which supports the foot or the shoe of the user and which is fixed to a distal part of the rigid sole at one end and free at the other end, and retention means for holding the foot or the shoe of the user on said blade.

4. The slipper as claimed in claim 3, wherein the flexible blade has a flexural stiffness of between 1 Nm/rad and 45 Nm/rad.

5. The slipper as claimed in claim 1, wherein, with the shoe of the user being equipped with a male or female clip-on end, said means for fixing comprise a female or male clip-on member of complementary shape, integrally connected to the distal end part of the rigid sole and movable in rotation about said pivot axis.

6. The slipper as claimed in claim 1, including abutment means designed to limit the rotation about the pivot axis to an adjustable value of less than 30° with respect to the rigid sole.

7. The slipper as claimed in claim 1, comprising a first pressure sensor fixed at the distal part of the rigid sole, and a second pressure sensor situated at a proximal part of said sole, and measuring and calculating means for detecting the lifting of the foot or of the shoe of the user from said rigid sole on the basis of the measurements carried out on said sensors.

8. The slipper as claimed in claim 1, comprising shock-absorbing means designed to absorb the heel strike.

9. An exoskeleton for lower limbs, comprising two articulated legs, each having a thigh, a shank and means for connecting the lower end of the shank to a slipper as claimed in claim 1, wherein said means for connecting to the slipper comprise an elongate component of a defined length, articulated in rotation at one end with said lower end of the shank and rigidly fixed at the other end to form an angle with the rigid sole.

10. The exoskeleton as claimed in claim 9, comprising a connecting member between the upper ends of the legs on which it is articulated, said connecting member being able to be positioned at the level of the pelvis of the user, and means for commanding the actuation of the articulated legs as a function of measuring and calculating means which are designed to detect the lifting of the foot or of the shoe of the user from said rigid sole on the basis of the measurements carried out on one or more sensors, such that the lifting of the foot of the user is anticipated.

11. The exoskeleton as claimed in claim 10, comprising calculating means for limiting the forces of the user on the exoskeleton in the position of lifting of the foot of the user, during the setting in motion of the leg.

12. A method for using an exoskeleton comprising slippers provided with a rigid sole and means for fixing the feet of a user on said sole, wherein said means for fixing are movable in rotation about a pivot axis integrally connected to the sole in the area of the distal end thereof, so as to allow the heel of the shoe of the user to lift from the rigid sole when walking, the method comprising:
acquiring, as a user is in the process of walking, the pressure values and/or the angle values of the heels of the user with respect to the ground continuously and/or at regular time intervals, and
when the pressure and/or the angle detected drop in value below reference values that are introduced beforehand into a calculating means and correspond to a foot bearing on the ground, generating a command anticipating the movement of the leg.

13. The method as claimed in claim 12, wherein, on the basis of the detection of the intention of movement corresponding to the drop below said one or more determined values, an automatic function mode of the legs of the exoskeleton is initiated using a previously recorded gait pattern.
